(19) United States
(12) Patent Application Publication
(10) Pub. No.: US 2020/0178783 A1
(43) Pub. Date: Jun. 11, 2020

(54) APPARATUS AND SYSTEM FOR GUIDING A CLEANING IMPLEMENT AND A METHOD OF USE THEREOF

(71) Applicant: ASP Global Manufacturing GmbH, Schaffhausen (CH)

(72) Inventors: Astor Suriano, Irvine, CA (US); Eshan Shameli, Irvine, CA (US); Navid Omidbakhsh, Irvine, CA (US)

(21) Appl. No.: 16/667,038

(22) Filed: Oct. 29, 2019

Related U.S. Application Data
(60) Provisional application No. 62/775,550, filed on Dec. 5, 2018.

(51) Int. Cl. A61B 1/12; A61L 2/16

(52) U.S. Cl. CPC A61B 1/122; A61L 2202/24; A61L 2/16

(57) ABSTRACT

An apparatus and a system for guiding a cleaning implement and a method of use thereof are provided. The apparatus comprises a body and a guide. The body comprises a surface configured to be received by a valve port of a medical device. The guide comprises a first guide port, a second guide port, and a conduit. The first guide port is disposed at a first position on the surface of the body and the second guide port is disposed at a second position on the surface of the body that is different from the first position. The second position is configured to align the second guide port with a lumen of the medical device. The conduit extends through the body from the first guide port to the second guide port. The conduit comprises a contour configured to guide the cleaning implement between the first guide port and the lumen.

### CROSS REFERENCE TO RELATED APPLICATIONS

The present application claims priority under 35 U.S.C. § 120 to co-pending U.S. provisional Patent Application Ser. No. 62/775,550, filed Dec. 5, 2018, which is incorporated by reference herein in its entirety.

### FIELD

The present disclosure relates to an apparatus and a system for guiding a cleaning implement, such as into a lumen of a medical device, and a method of use thereof.

### BACKGROUND

Medical devices, such as endoscopes, are used in numerous procedures in the medical field. These devices are as varied as the procedures themselves. After a medical device is used, the medical device is treated (e.g., cleaned, disinfected, and/or sterilized) in order to prepare the medical device for its next use. Ensuring the medical device is properly cleaned prior its next use can prevent the risk of cross contamination and the spread of disease.

### SUMMARY

In one aspect, an apparatus for guiding a cleaning implement into a lumen of a medical device is provided. The apparatus comprises a body and a guide. The body comprises a surface and the body is configured to be received by a valve port of the medical device. The valve port of the medical device is in fluid communication with the lumen of the medical device. The guide comprises a first guide port, a second guide port, and a conduit. The first guide port is disposed at a first position on the surface of the body and configured to receive the cleaning implement. The second guide port is disposed at a second position on the surface of the body that is different from the first position. The second guide port is configured to receive the cleaning implement. The second position is configured to align the second guide port with the lumen. The conduit extends through the body from the first guide port to the second guide port. The conduit comprises a contour configured to guide the cleaning implement between the first guide port and the lumen.

In another aspect, a system comprising a cleaning implement, an apparatus for guiding the cleaning implement into a lumen of a medical device, and an automated cleaning module is provided. The apparatus comprises a body and a guide. The body comprises a surface and the body is configured to be received by a valve port of the medical device. The valve port of the medical device is in fluid communication with the lumen of the medical device. The guide comprises a first guide port, a second guide port, and a conduit. The first guide port is disposed at a first position on the surface of the body and configured to receive the cleaning implement. The second guide port is disposed at a second position on the surface of the body that is different from the first position. The second guide port is configured to receive the cleaning implement. The second position is configured to align the second guide port with the lumen. The conduit extends through the body from the first guide port to the second guide port. The conduit comprises a contour configured to guide the cleaning implement between the first guide port and the lumen. The automated cleaning module is operatively engaged with the cleaning implement. The brushing module is configured to move the cleaning implement through the conduit.

In yet another aspect, a system comprising a medical device and an apparatus for guiding a cleaning implement into a lumen of the medical device is provided. The apparatus comprises a body and a guide. The body comprises a surface and the body is configured to be received by a valve port of the medical device. The valve port of the medical device is in fluid communication with the lumen of the medical device. The guide comprises a first guide port, a second guide port, and a conduit. The first guide port is disposed at a first position on the surface of the body and configured to receive the cleaning implement. The second guide port is disposed at a second position on the surface of the body that is different from the first position. The second guide port is configured to receive the cleaning implement. The second position is configured to align the second guide port with the lumen. The conduit extends through the body from the first guide port to the second guide port. The conduit comprises a contour configured to guide the cleaning implement between the first guide port and the lumen. The medical device comprises a valve port in fluid communication with the lumen. The apparatus is disposed within the valve port of the medical device.

In a further aspect, a method for cleaning a lumen of a medical device is provided. An apparatus for guiding a cleaning implement into a lumen of a medical device is introduced into a valve port of the medical device. The valve port is in fluid communication with the lumen of the medical device. The apparatus comprises a body and a guide. The body comprises a surface configured to be received by a valve port of the medical device. The valve port of the medical device is in fluid communication with the lumen of the medical device. The guide comprises a first guide port, a second guide port, and a conduit. The first guide port is disposed at a first position on the surface of the body and configured to receive the cleaning implement. The second guide port is disposed at a second position on the surface of the body that is different from the first position. The second guide port is configured to receive the cleaning implement. The second position is configured to align the second guide port with the lumen. The conduit extends through the body from the first guide port to the second guide port. The conduit comprises a contour configured to guide the cleaning implement between the first guide port and the lumen. The cleaning implement is introduced into the conduit of the apparatus and the lumen of the medical device. The lumen of the medical device is cleaned with the cleaning implement.

It is understood that the inventions described in this specification are not limited to the examples summarized in this Summary. Various other aspects are described and exemplified herein.

### BRIEF DESCRIPTION OF THE DRAWINGS

The features and advantages of the examples, and the manner of attaining them, will become more apparent and the examples will be better understood by reference to the following description taken in conjunction with the accompanying drawings, wherein:

FIG. 1A is a perspective view of a non-limiting example of an apparatus for guiding a cleaning implement;

FIG. 1B is a front-elevation view of a non-limiting example of the apparatus of FIG. 1A;

FIG. 2 is a perspective view of a medical device to be cleaned, in the form of an endoscope;

FIG. 3 is a front perspective view of a non-limiting example of a system comprising the apparatus of FIG. 1A and the first portion of the endoscope in a first configuration;

FIG. 4 is a front perspective view of a non-limiting example of the system comprising the apparatus of FIG. 1A and the first portion of the endoscope in a second configuration;

FIG. 5A is a front perspective view of a non-limiting example of the system comprising the apparatus of FIG. 1A and the first portion of the endoscope in a third configuration;

FIG. 5B is a bottom perspective view of the system of FIG. 5A;

FIG. 5C is a front elevation view of the system of FIG. 5A; and

FIG. 6 is a front elevational view of a non-limiting example of a system comprising the apparatus of FIG. 1A and the first portion of the endoscope in the third configuration, and the cleaning implement in the form of a brush.

Corresponding reference characters indicate corresponding parts throughout the several views. The exemplifications set out herein illustrate certain embodiments, in one form, and such exemplifications are not to be construed as limiting the scope of the examples in any manner.

### DETAILED DESCRIPTION

Certain exemplary aspects of the present disclosure will now be described to provide an overall understanding of the principles of the structure, function, manufacture, and use of the devices and methods disclosed herein. One or more examples of these aspects are illustrated in the accompanying drawings. Those of ordinary skill in the art will understand that the devices and methods specifically described herein and illustrated in the accompanying drawings are non-limiting exemplary aspects and that the scope of the various examples of the present invention is defined solely by the claims. The features illustrated or described in connection with one exemplary aspect may be combined with the features of other aspects. Such modifications and variations are intended to be included within the scope of the present invention.

Reference throughout the specification to “various examples,” “some examples,” “one example,” or “an example”, or the like, means that a particular feature, structure, or characteristic described in connection with the example is included in at least one example. Thus, appearances of the phrases “in various examples,” “in some examples,” “in one example”, or “in an example”, or the like, in places throughout the specification are not necessarily all referring to the same example. Furthermore, the particular features, structures, or characteristics may be combined in any suitable manner in one or more examples. Thus, the particular features, structures, or characteristics illustrated or described in connection with one example may be combined, in whole or in part, with the features, structures, or characteristics of one or more other examples without limitation. Such modifications and variations are intended to be included within the scope of the present examples.

In this specification, unless otherwise indicated, all numerical parameters are to be understood as being prefaced and modified in all instances by the term “about”, in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Also, any numerical range recited herein includes all sub-ranges subsumed within the recited range. For example, a range of “1 to 10” includes all sub-ranges between (and including) the recited minimum value of 1 and the recited maximum value of 10, that is, having a minimum value equal to or greater than 1 and a maximum value equal to or less than 10. Any maximum numerical limitation recited in this specification is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited. All such ranges are inherently described in this specification such that amending to expressly recite any such sub-ranges would comply with the requirements of 35 U.S.C. § 112 and 35 U.S.C. § 132(a).

The grammatical articles “a”, “an”, and “the”, as used herein, are intended to include “at least one” or “one or more”, unless otherwise indicated, even if “at least one” or “one or more” is expressly used in certain instances. Thus, the articles are used herein to refer to one or more than one (i.e., to “at least one”) of the grammatical objects of the article. Further, the use of a singular noun includes the plural, and the use of a plural noun includes the singular, unless the context of the usage requires otherwise.

A device can undergo a treatment process to prevent cross-contamination and the spread of disease. As used herein, a “treatment process” may be a cleaning process, a disinfecting process, a sterilization process, the like, and combinations thereof. A treatment process may be either manual, automated, or some combination thereof, and may utilize a treatment agent. As used herein, a “treatment agent” can comprise at least one of a cleaning agent, a disinfectant, and a sterilant. As used herein a “cleaning process” means a treatment process employing a cleaning agent that removes and/or eliminates debris such as, for example, a dirt, a dust, a particle, an oil, a protein, a carbohydrate, and the like. As used herein, a “cleaning agent” means a type of treatment agent that removes and/or eliminates debris during a cleaning process such as, for example, a surfactant and/or a detergent.

A disinfecting process and a sterilization process can remove and/or eliminate a bioburden from a device. A bioburden may be, for example, a bacterium (e.g., mycobacterium, bacterial spores), an archaeon, a eukaryote, a virus, a fungus, and/or other forms of biological agents. Bacterial spores (e.g., endospores) are a form of bacteria which are dormant and highly resistive to physical and chemical degradation. As used herein, a “disinfecting process” means a treatment process that substantially removes a bioburden except for bacterial spores. As used herein, “substantially remove” means that at least 99% of the bioburden has been removed from the device such as, for example, at least 99.9% of the bioburden, at least 99.99% of the bioburden, at least 99.999% of the bioburden, or at least 99.9999% of the bioburden has been removed from the device. As used herein, a “sterilization process” means a treatment process which substantially removes a bioburden including bacterial spores. The sterilization process may include, for example, the addition of heat, freezing, a sterilant, irradiation, pressure, and combinations thereof. The sterilant may comprise a chemical capable of sterilization. The disinfection process may include, for example, the addition of heat, a disinfectant, irradiation, pressure, and combinations thereof. The disinfectant may comprise a chemical capable of disinfection.

As used herein, a “cleaning implement” means any cleaning device that can be applied to a surface of a lumen to clean the surface thereof. The cleaning implement can be or comprise, for example, at least one of a brush, a wire, a tube, a catheter, and a flexible scraper. As used herein, a “brush” is one form of cleaning implement and means a device comprising a shank and bristles set into at least a portion of the shank. In various examples, the bristles may comprise hair and/or wire. The shank may comprise, for example, a round solid shaft or two or more twisted wires. The bristles may radially extend from a portion of the shank to form a brush head comprising a generally cylindrical shape. For example, the brush head may comprise a partially cylindrical shape. A brush can be used for cleaning processes, such as, for example, scrubbing, brushing, rubbing, scouring, or washing the surface of the device. In various examples, the brush may be a tube brush.

Lumens in a medical device, such as those in an endoscope, may be difficult to clean because debris may remain in the lumen after use that is difficult to access by traditional cleaning processes. The lumens for gas and water in the control head of the endoscope may not be accessible to a brush due to their position inside of valve ports on the control head. The debris remaining in these lumens may impede a subsequent disinfection process and/or sterilization process. Thus, an apparatus and a system for guiding a cleaning implement into a lumen of a medical device and a method of use thereof according to the present disclosure are provided. The apparatus, system, and method can facilitate efficient cleaning of a lumen of a medical device and can improve the effectiveness of a post-cleaning disinfection process and/or sterilization process.

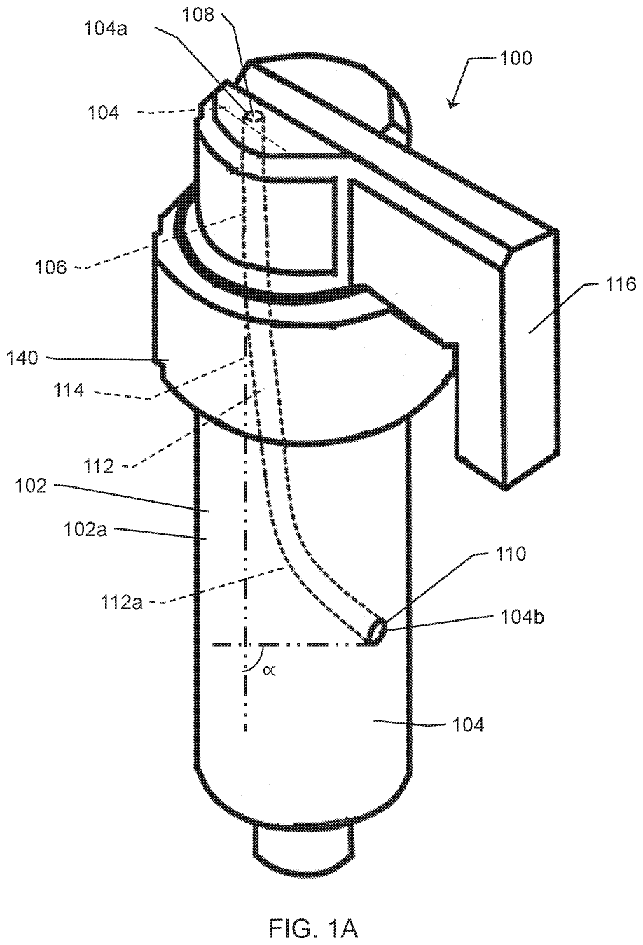

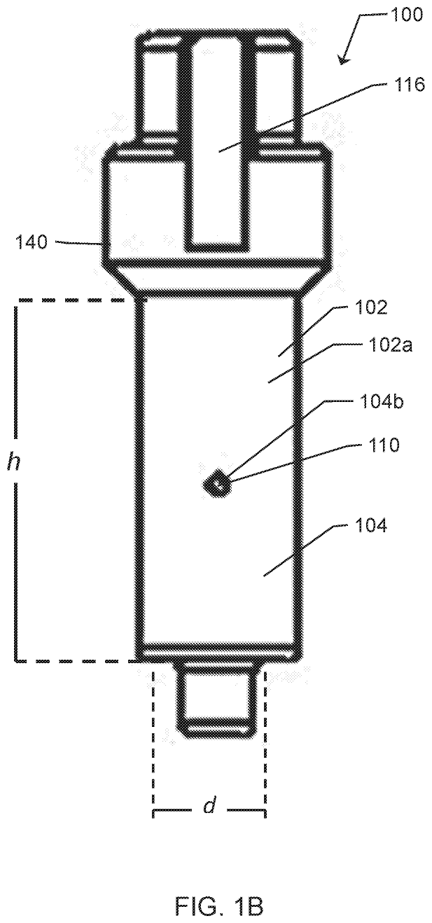

As illustrated in FIGS. 1A-B, an apparatus 100 for guiding a cleaning implement into a lumen of a medical device is provided. The apparatus comprises a body 102 comprising a surface 104 configured to be received by a valve port of the medical device to be cleaned (e.g., endoscope). For example, the body 102 can comprise a generally circular cross section which may be received by a valve port of the medical device to be cleaned. As shown in FIG. 1E, the generally circular cross section can have a diameter, d, of at least 4 mm, such as, for example, at least 5 mm, or at least 10 mm. The generally circular cross section can have a diameter, d, of 20 mm or less, such as, for example, 16 mm or less, or 10 mm or less. The generally circular cross section can have a diameter, d, in a range of 4 mm to 20 mm, such as, for example, 5 mm to 16 mm.

As illustrated in FIGS. 1A-B, the body 102 may comprise a guide 106 comprising a first guide port 108 and a second guide port 110. The guide 106 can be suitable to receive a cleaning implement and provide the cleaning implement to a lumen within a valve port of a medical device with minimal, if any, kinking of a shank of the cleaning implement.

The first guide port 108 may be disposed at a first position 104a on the surface 104 of the body 102 and may be configured to receive the cleaning implement. The first guide port 108 can receive the cleaning implement while the body 102 can be received by the valve port of the medical device to be cleaned. The first guide port 108 can comprise at least one of a taper, a chamfer, and a radius to facilitate entry of the cleaning implement into the first guide port 108. The position, orientation, and shape of the first guide port 108 can be structured to enable movement of the cleaning implement into and through the first guide port 108 and into a conduit 112 of the guide 106. The conduit 112 may extend through the body 102 from the first guide port 108 to a second guide port 110. The second guide port 110 may be disposed at a second position 104b on the surface 104 of the body 102 that can be different from the first position 104a. The second position 104b can be configured to align the second guide port 110 with a lumen of the medical device. The second guide port 110 can be configured to receive the cleaning implement. The position, orientation, and shape of the second guide port 110 can be configured to enable movement of the cleaning implement into and through the second guide port 110 and into a lumen of a medical device. For example, the second guide port 110 can be suitable to align with a lumen of a medical device.

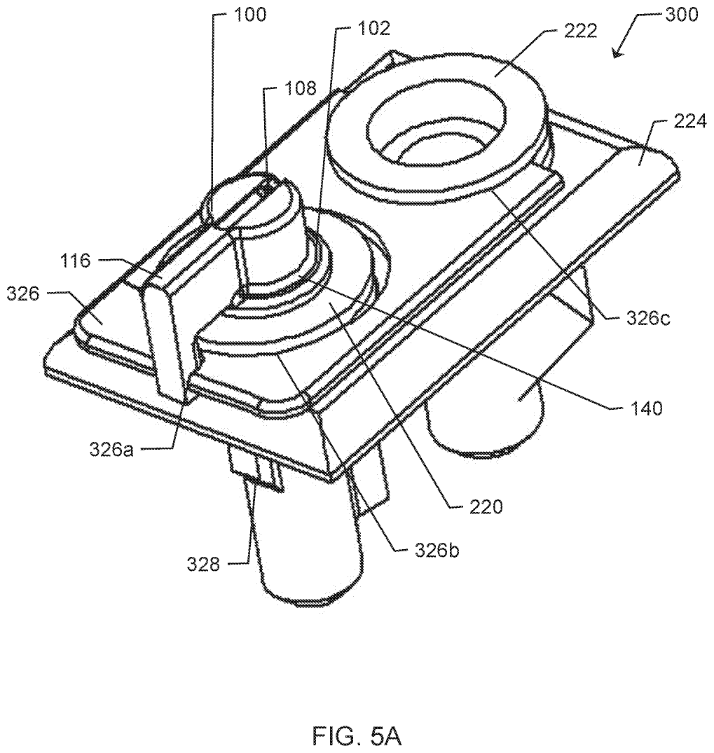

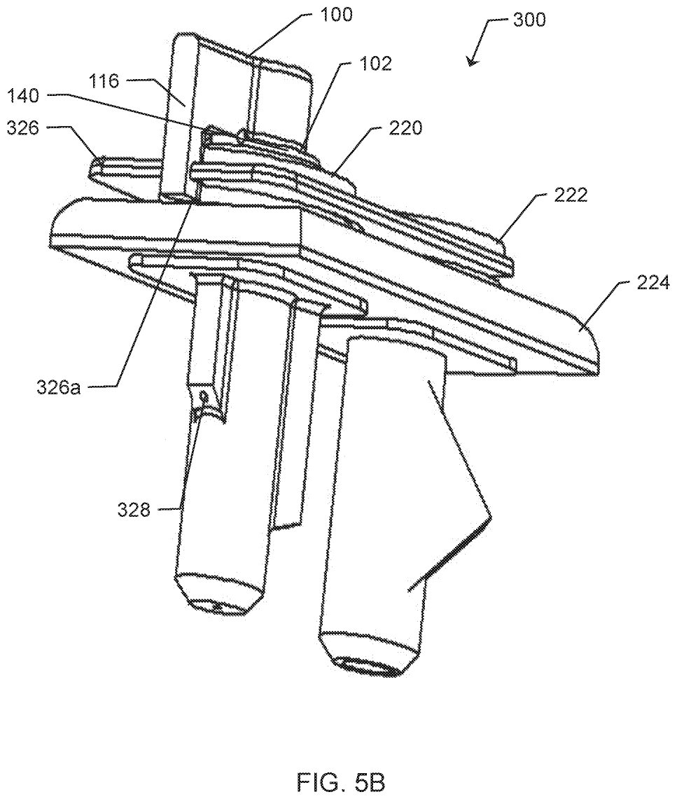

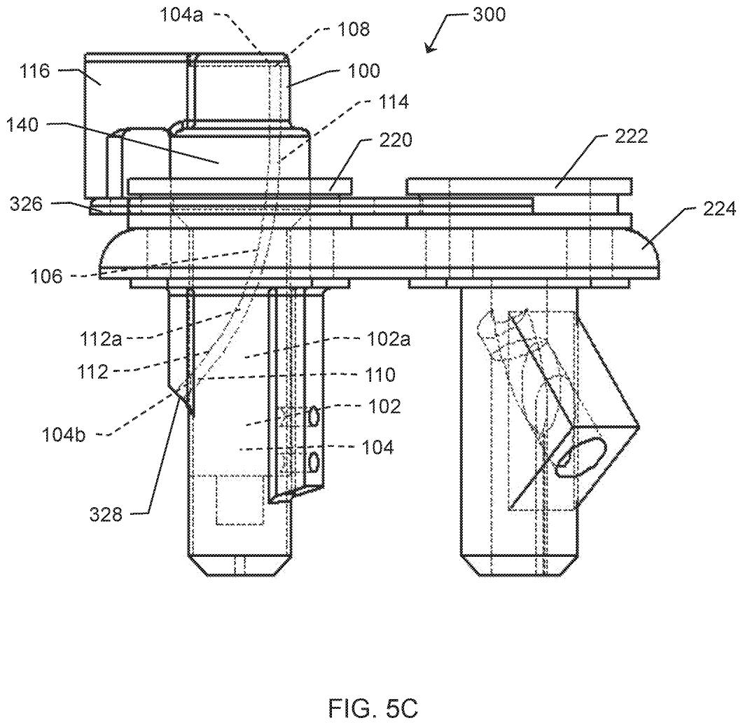

The body 102 can have a portion 102a (e.g., shaft portion) that can be received by a cavity of a valve port of a medical device. For example, the portion 102a can have a penetration height, h, which can be a sufficient length such that the second guide port 110 can align with the lumen of a medical device, but may be less than the depth of the cavity of the valve port so the portion 102a can be received by the cavity of the valve port as shown and further described in reference to FIG. 5C herein. The penetration height, h, can be at least 4 mm, such as, for example, at least 5 mm or at least 20 mm. The penetration height, h, can be 35 mm or less, such as, for example, 30 mm or less or 20 mm or less. The penetration height, h, may be in a range of 5 mm to 35 mm, such as, for example, 5 mm to 30 mm.

The body 102 can comprise a protrusion 140 suitable to engage a surface of a valve port and prevent further entry of the body 102 when the body 102 is inserted into the valve port of the medical device. The protrusion 140 can be disposed adjacent to the portion 102a.

As illustrated in FIG. 1A, the conduit 112 can comprise a contour 114 configured to guide the cleaning implement between the first guide port 108 and a lumen of the medical device. For example, the first guide port 108 may be radially offset from the second guide port 110 such that the cleaning implement entering the first guide port 108 may have to change direction to enter into the second guide port 110 and exit the guide 106. For example, the conduit 112 can comprise a section 112a that forms a bend angle, a, suitable to limit and, in various examples, prevent the shank of the cleaning implement from kinking while traversing through the conduit 112 from the first guide port 108 to the second guide port 110. For example, the bend angle, a, can be at least 30 degrees, such as, for example, at least 40 degrees, at least 50 degrees, at least 60 degrees, at least 70 degrees, or at least 80 degrees. The bend angle, a, can be 90 degrees or less, 80 degrees or less, 70 degrees or less, 60 degrees or less, 50 degrees or less, or 40 degrees or less. The bend angle, a, may be in a range of 30 degrees to 90 degrees, such as, for example, 30 degrees to 80 degrees or 40 degrees to 70 degrees. The first guide port 108 may not be radially offset from the second guide port 110 such that the conduit 112 is substantially linear.

The contour 114 can comprise a generally cylindrical shape, such as, for example, a tubular shape extending from the first guide port 108 to the second guide port 110. The contour 114 can comprise a cross-sectional diameter of at least 0.5 mm, such as, for example, at least 1 mm or at least 2 mm. The contour 114 can comprise a cross-sectional diameter of 3 mm or less, such as, for example, 2 mm or less or 1 mm or less. The contour 114 can comprise a cross-sectional diameter in a range of 0.5 mm to 3 mm.

The apparatus 100 can comprise an alignment member 116 configured to indicate the second guide port 110 is aligned with a lumen of the medical device and/or to facilitate alignment of the second guide port 110 with the lumen of the medical device. The alignment member 116 can comprise at least one of a protrusion, a keyway, and a mark (e.g., a label) configured to indicate and/or facilitate alignment of the second guide port 110 with a lumen. The alignment member 116 can be aligned with a feature of a medical device and/or other alignment unit. When the body 102 is received by a valve port of a medical device, the second guide port 110 may not be observable and the alignment member 116 may be observable. Thus, the alignment member 116 can be utilized as a reference for the position of the second guide port 110.

The first guide port 108 can be formed with a fitting, such as, for example, a luer fitting or a compression fitting. The fitting can facilitate engagement of the first guide port 108 to tubing suitable to receive and guide the cleaning implement into the first guide port 108. For example, the first guide port 108 can be connected to a first fitting and the tubing can be connected to a second fitting. The first fitting can engage with the second fitting to create fluid communication between the tubing and the first guide port 108. In various examples, a tube can be directly connected to the first guide port 108. Regardless of the connection type, the tube can be configured to receive the cleaning implement and to direct the cleaning implement into the first guide port 108. The fittings can facilitate quick connections between the first guide port 108 and the tubing to efficiently provide the cleaning implement to the guide 106.

The apparatus 100 can comprise at least two guides including guide 106. For example, each guide can be suitable to receive the cleaning implement and provide the cleaning implement to the lumen within a valve port of a medical device with minimal, if any, kinking of a shank of the cleaning implement. In various examples, at least two lumens in the valve port can be cleaned with the use of at least two guides. Each guide port may be equipped with a fitting. Thus, the cleaning implements can be quickly aligned with the guide on each guide port utilizing the tubing.

The position and/or orientation of the apparatus 100 can be changed to clean at least two lumens in a valve port with a single guide.

The apparatus 100 can be formed of any material suitable for use with a cleaning process and in various examples, a disinfection process and/or a sterilization process. For example, the apparatus can comprise at least one of a plastic, a metal, a glass, and a ceramic.

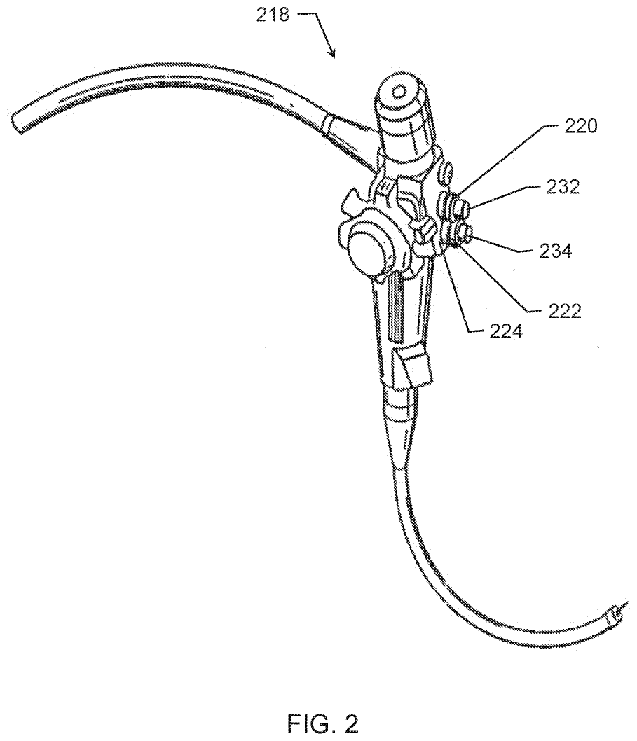

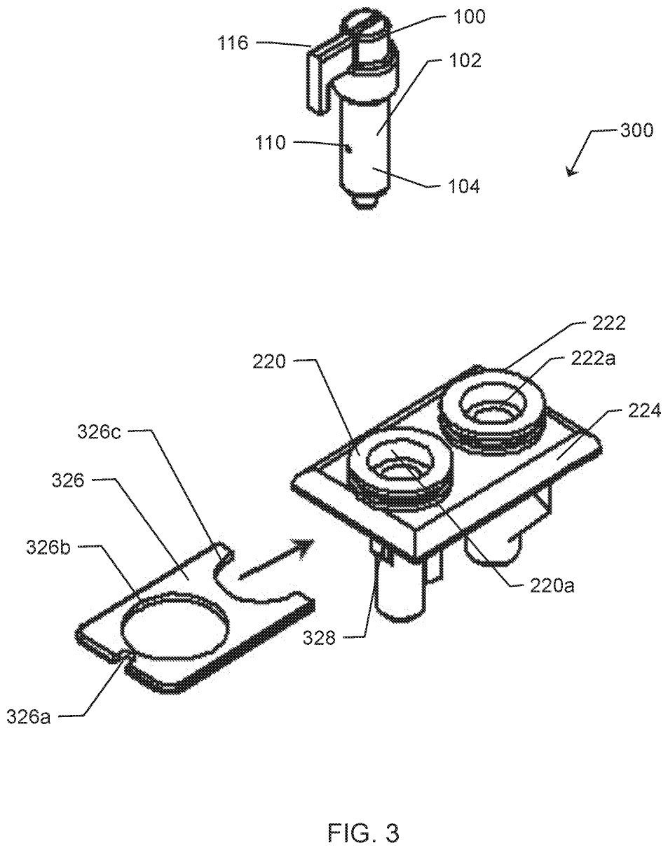

As illustrated in FIG. 2, an endoscope 218 is provided to illustrate one application of the apparatus, system, and method of the present disclosure. The endoscope 218 can comprise a first valve port 220 comprising a first valve unit 232. The endoscope 218 can comprise a second valve port 222 comprising a second valve unit 234. It is contemplated that the endoscope 218 can comprise at least three valve ports, and the teachings of the present disclosure can be applied to a multiplicity of valve ports. Each valve port can be at least one of a gas valve port (e.g., pressure or vacuum) and a water vale port, and each valve can be at least one of a gas valve and a water valve. The first and second valve ports 220, 222 can be positioned at a first portion 224 of the endoscope 218, such as, for example, a control head of the endoscope 218. Each valve port 220, 222 can be in fluid communication with a lumen of the endoscope 218 and in various examples at least two lumens of the endoscope 218. Each lumen can comprise a diameter ranging from 0.5 mm to 3 mm. The diameter of the lumen can depend on the manufacture of the endoscope and the purpose of the lumen (e.g., dispensing or drawing water, air, or carbon dioxide, or pulling a vacuum). As shown in FIG. 3, the first valve port 220 can be in fluid communication with lumen 328 and, as shown in FIG. 5C and as further described herein.

Turning again to FIGS. 2 and 3, the first valve unit 232 can be removed from the first valve port 220 of the endoscope 218 to expose a cavity 220a of the first valve port 220. The second valve unit 234 can be removed from the second valve port 222 of the endoscope 218 to expose a cavity 222a of the second valve port 222. The cavities 220a, 222a can be suitable to receive the body 102 of the apparatus 100.

As illustrated in FIG. 3, a system 300 comprising the apparatus 100 and the first portion 224 of the endoscope 218 is provided in a first configuration wherein the apparatus 100 is outside of the first valve port 220 and the body 102 is positioned to be inserted therein. In the first configuration, the apparatus 100 can be aligned with the first valve port 220. The surface 104 of the body 102 of the apparatus 100 can be configured to be received by the valve port 220. The body 102 can be any shape that can be suitably received by the valve port 220. For example, the body 102 can have a generally cylindrical shape and the valve port 220 can have a complementary, generally cylindrical shape. The body 102 can have a diameter less than a diameter of the valve port 220 such that the body 102 can traverse into the first valve port 220. For example, the diameter of the body 102 can be less than the diameter of the first valve port 220 by at least 0.01 mm, such as, for example, at least 0.1 mm or at least 1 mm. The diameter of the body 102 can be less than the diameter of the first valve port 220 by 2 mm or less, such as, for example, 1 mm or less or 0.1 mm or less. The diameter of the body 102 can be less than the diameter of the first valve port 220 in a range of 0.01 mm to 2 mm.

The system 300 can further comprise an alignment unit 326. The alignment unit 326 can comprise a keyway 326a suitable to engage the alignment member 116 of the apparatus 100, for example, following insertion of the body 102 into the first valve port 220. The alignment unit 326 can facilitate alignment of the second guide port 110 with the lumen 328. The alignment unit 326 can be configured to engage the first portion 224 of the endoscope 218. For example, at least one of the first valve port 220 and the second valve port 222 can be engaged by at least one of a first alignment receiver 326b and a second alignment receiver 326c, respectively. In the first configuration, the alignment unit 326 can be disposed separate from the first portion 224.

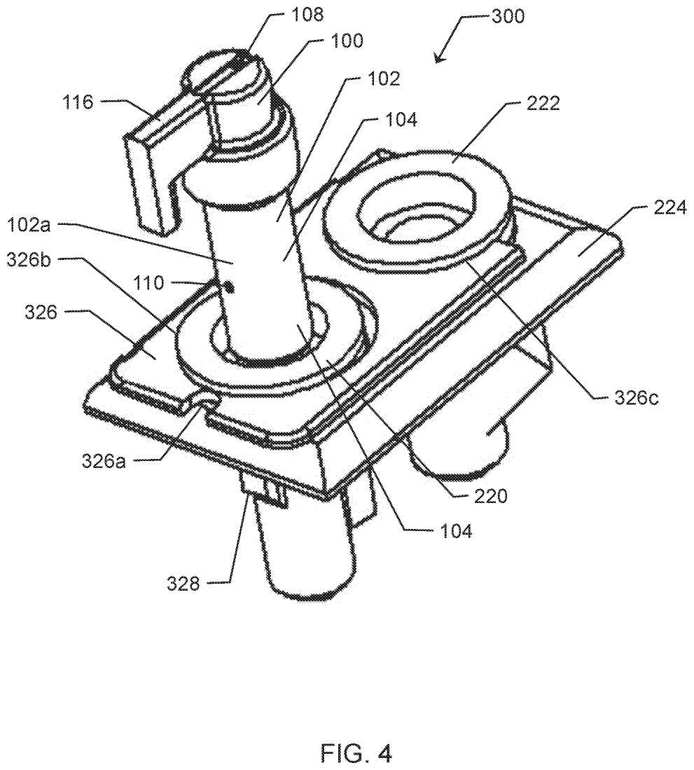

FIG. 4 illustrates the system 300 of the present disclosure in a second configuration wherein at least a portion of the body 102 is at least partially inserted into the valve port 220. In the second configuration, the body 102 can be at least partially received by the first valve port 220 and the alignment member 116 of the apparatus 100 can be at least partially aligned with the keyway 326a of the alignment unit 326. The alignment unit 326 can be positioned in engagement with the first portion 224. Namely, the first alignment receiver 326b can receive and engage the first valve port 220 and the second alignment receiver 326c can receive and engage the second valve port 222. When the alignment unit 326 is engaged with the first portion 224, the keyway 326a can be in a position suitable to engage the alignment member 116.

FIGS. 5A-C, illustrates the system 300 of the present disclosure in a third configuration wherein the apparatus 100 is disposed within the first valve port 220 of the endoscope 218. The entry of the body 102 into the first valve port 220 can be limited by at least one of the alignment member 116 and the protrusion 140. The alignment member 116 can engage the keyway 326a and responsive to the engagement the second guide port 110 (FIGS. 3 and 4) can be aligned with the lumen 328. The engagement of the alignment member 116 with the keyway 326a can provide a visual indication for an operator that the second guide port 110 is aligned with the lumen 328. The first guide port 108 can be exposed and the second guide port 110 may not be exposed when the body 102 is received by the cavity 220a.

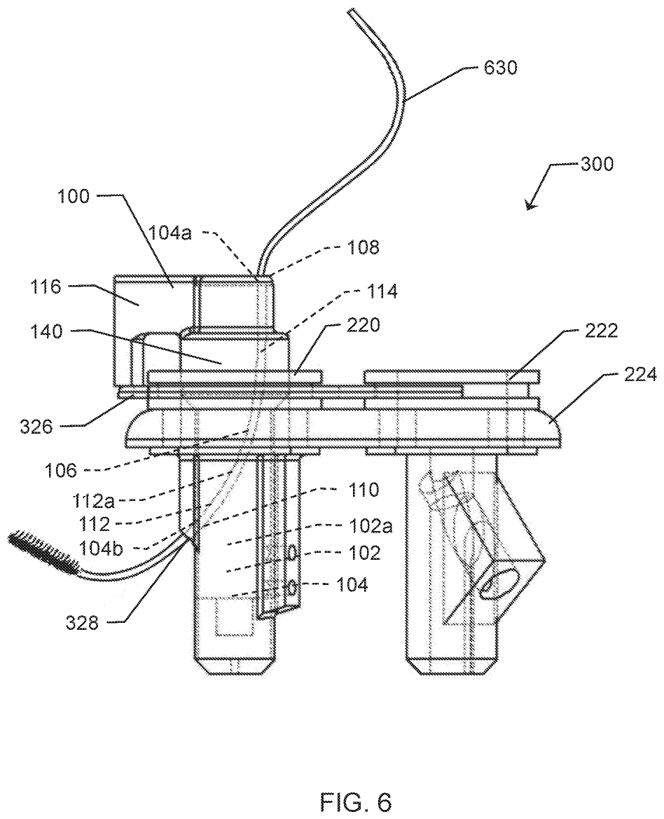

As illustrated in FIG. 6, the cleaning implement 630, such as a brush, can be inserted into and received by the guide 106 of the apparatus 100 when the system 300 is in the third configuration. For example, and by way of illustration only, the cleaning implement 630 can be introduced into the first guide port 108 and traverse through the conduit 112 and the second guide port 110 into the lumen 328. Mechanical force applied by the cleaning implement 630 to the lumen 328 can reduce and/or eliminate debris from the lumen 328 (e.g., clean the lumen). For example, bristles of a brush can contact a surface of the lumen 328 and mechanically remove and/or eliminate debris therefrom. Detergent can be introduced to the lumen 328 to aid in the mechanical removal and/or elimination of debris.

The cleaning implement 630, can be operatively engaged to an automated cleaning module (not shown). The automated cleaning module can be configured to move the cleaning implement 630 through the conduit 112 and into the lumen 328 to facilitate a cleaning process and application of mechanical force to the lumen 328 in order to reduce and/or eliminate debris from the lumen 328. The automated cleaning module can facilitate traversal of the cleaning implement 630 through the lumen 328 in a substantially linear motion and/or a substantially radially motion (e.g., spinning). It is contemplated that other mechanical operations such as vibration, pulsation, sonic action, and the like may also be employed to assist the cleaning implement in the cleaning operation.

A treatment apparatus can comprise the automated cleaning module. For example, the treatment apparatus can comprise at least one of a high-level disinfection device and a sterilizer. For example, the sterilizer can be a STERRAD® brand of sterilizers manufactured by Advanced Sterilization Products, of Irvine, Calif., a division of Ethicon, Inc. of Somerville, N.J. The high-level disinfection apparatus can be an EVOTECH® ECR brand of high-level disinfection apparatus manufactured by Advanced Sterilization Products, of Irvine, Calif., a division of Ethicon, Inc. of Somerville, N.J. The automated cleaning module can comprise cleaning implements or be connected to cleaning implements introduced into the lumens in order to facilitate cleaning of the lumens. For example, the automated cleaning module can comprise a tubing comprising a cleaning implement within the cavity of the tubing. For example, the tubing can be connected to the first guide port 108 of the apparatus 100 and the automated cleaning module can introduce the cleaning implement 630 into the guide 106 and into the lumen 328. The cleaning process with the cleaning implement 630 in the lumen 328 can occur prior to a treatment cycle in a treatment apparatus or simultaneously with the treatment cycle in the treatment apparatus.

A method for cleaning a lumen of a medical device is provided herein. The apparatus for guiding a cleaning implement according the present disclosure can be introduced into a lumen in fluid communication with a valve port of the medical device. In various examples, a valve (e.g., gas valve or water valve) can be removed from the valve port of the medical device prior to introducing the apparatus for guiding a cleaning implement according to the present disclosure. A cleaning implement can be introduced into the conduit of the apparatus for guiding the cleaning implement according to the present disclosure and the lumen of the medical device. The lumen of the medical device can be cleaned with the cleaning implement. In various embodiments, the medical device can be contacted with a detergent. For example, the medical device can be sprayed with detergent, detergent can be introduced into the lumen of the medical device, and/or the medical device can be submerged in detergent. The detergent can improve the efficiency of the removal and/or elimination of debris by the cleaning implement.

In various examples, the apparatus and the medical device can be introduced into a treatment apparatus. The treatment apparatus can be operated to clean the lumen of the medical device with the cleaning implement. For example, the treatment apparatus can comprise an automated cleaning module operatively engaged to the cleaning implement. The automated cleaning module can move the cleaning implement to mechanically remove and/or eliminate debris from a surface of the lumen. The automated cleaning can occur prior to or simultaneously with a treatment cycle with the treatment apparatus.

Lumens in a medical device, such as those in an endoscope, are difficult to clean because debris remains in the lumen after use that is difficult to access by traditional cleaning processes. The lumens for gas and water in the control head of the endoscope may not be accessible to a brush due to their position inside of valve ports on the control head. The debris remaining in these lumens may impede a subsequent disinfection process and/or sterilization process. The apparatus, system, and method for guiding a cleaning implement into a lumen of a medical device as provided herein that can facilitate efficient cleaning of a lumen of an endoscope and can improve the effectiveness of a post-cleaning disinfection process and/or sterilization process.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

Although various examples have been described herein, many modifications, variations, substitutions, changes, and equivalents to those examples may be implemented and will occur to those skilled in the art. Also, where materials are disclosed for certain components, other materials may be used. It is therefore to be understood that the foregoing description and the appended claims are intended to cover all such modifications and variations as falling within the scope of the disclosed examples. The following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Various aspects of the invention according to the present disclosure include, but are not limited to, the aspects listed in the following numbered clauses.
1. An apparatus for guiding a cleaning implement into a lumen of a medical device, the apparatus comprising:
   a body comprising a surface, the body is configured to be received by a valve port of the medical device, the valve port in fluid communication with the lumen; and
   a guide comprising:
      a first guide port disposed at a first position on the surface of the body and configured to receive the cleaning implement;
      a second guide port disposed at a second position on the surface of the body that is different from the first position, the second guide port configured to receive the cleaning implement, the second position configured to align the second guide port with the lumen; and
      a conduit extending through the body from the first guide port to the second guide port, the conduit comprising a contour configured to guide the cleaning implement between the first guide port and the lumen.
2. The apparatus of clause 1, wherein the body has a generally cylindrical shape and the body has a diameter less than a diameter of the valve port.
3. The apparatus of any one of clauses 1-2, wherein the body comprises a generally circular cross section.
4. The apparatus of clause 3, wherein the generally circular cross section has a diameter ranging from 4 mm to 20 mm.
5. The apparatus of any one of clauses 3-4, wherein the body has a penetration depth ranging from 5 mm to 35 mm.
6. The apparatus of any one of clauses 1-5, wherein the contour comprises a generally cylindrical shape.
7. The apparatus of clause 6, wherein the contour has a diameter ranging from 0.5 mm to 3 mm.
8. The apparatus of any one of clauses 1-7, wherein the contour further comprises a section forming a bend angle of at least 30 degrees.
9. The apparatus of any one of clauses 1-8, further comprising an alignment member configured to indicate the second guide port is aligned with the lumen.
10. The apparatus of any one of clauses 1-9, further comprising a fitting connected to the first guide port.
11. The apparatus of any one of clauses 1-10, further comprising a tube connected to the first guide port, the tube configured to receive the cleaning implement and to guide the cleaning implement into the first guide port.
12. The apparatus of any one of clauses 1-11, wherein the guide is a first guide and further comprising at least two guides including the first guide.
13. A system comprising:
   the apparatus and the cleaning implement of any one of clauses 1-12; and
   an automated cleaning module operatively engaged with the cleaning implement, the brushing module is configured to move the cleaning implement through the conduit.
14. The system of clause 13, further comprising a treatment apparatus comprising the automated cleaning module.
15. A system comprising:
   a medical device comprising a valve port in fluid communication with a lumen; and
   the apparatus of any one of clauses 1-12, the apparatus is disposed within the valve port of the medical device.
16. The system of clause 15, wherein the lumen comprises a diameter ranging from 0.5 mm to 3 mm.
17. The system of any one of clauses 15-16, wherein the valve port is disposed on a control head of the medical device.
18. The system of any one of clauses 15-17, wherein the valve port is at least one of a gas valve port and a water valve port.
19. A method for cleaning a lumen of a medical device, comprising:
   introducing the apparatus of any one of clauses 1-12 into a valve port of the medical device, the valve port is in fluid communication with the lumen of the medical device:
   introducing the cleaning implement into the conduit of the apparatus and the lumen of the medical device; and
   cleaning the lumen of the medical device with the cleaning implement.
20. The method of clause 19, further comprising contacting the medical device with a detergent.
21. The method of any one of clauses 19-20, further comprising removing a valve from the valve port of the medical device prior to the introducing of the apparatus.
22. The method of any one of clauses 19-21, further comprising:
   introducing the apparatus and the medical device in a treatment apparatus; and
   operating the treatment apparatus to clean the lumen of the medical device with the cleaning implement.

In summary, numerous benefits have been described which result from employing the concepts described herein. The foregoing description of the one or more examples has been presented for purposes of illustration and description. It is not intended to be exhaustive or limiting to the precise form disclosed. Modifications or variations are possible in light of the above teachings. The one or more examples were chosen and described in order to illustrate principles and practical application to thereby enable one of ordinary skill in the art to utilize the various embodiments and with various modifications as are suited to the particular use contemplated. It is intended that the claims submitted herewith define the overall scope.

While the present disclosure provides descriptions of various specific aspects for the purpose of illustrating various aspects of the present disclosure and/or its potential applications, it is understood that variations and modifications will occur to those skilled in the art. Accordingly, the invention or inventions described herein should be understood to be at least as broad as they are claimed, and not as more narrowly defined by particular illustrative aspects provided herein.

What is claimed is:

1. An apparatus for guiding a cleaning implement into a lumen of a medical device, the apparatus comprising:
a body comprising a surface, the body is configured to be received by a valve port of the medical device, the valve port in fluid communication with the lumen; and
a guide comprising:
a first guide port disposed at a first position on the surface of the body and configured to receive the cleaning implement;
a second guide port disposed at a second position on the surface of the body that is different from the first position, the second guide port configured to receive the cleaning implement, the second position configured to align the second guide port with the lumen; and
a conduit extending through the body from the first guide port to the second guide port, the conduit comprising a contour configured to guide the cleaning implement between the first guide port and the lumen.

2. The apparatus of claim 1, wherein the body has a generally cylindrical shape and the body has a diameter less than a diameter of the valve port.

3. The apparatus of claim 1, wherein the body comprises a generally circular cross section.

4. The apparatus of claim 3, wherein the generally circular cross section has a diameter ranging from 4 mm to 20 mm.

5. The apparatus of claim 3, wherein the body has a penetration depth ranging from 5 mm to 35 mm.

6. The apparatus of claim 1, wherein the contour comprises a generally cylindrical shape.

7. The apparatus of claim 6, wherein the contour has a diameter ranging from 0.5 mm to 3 mm.

8. The apparatus of claim 1, wherein the contour further comprises a section forming a bend angle of at least 30 degrees.

9. The apparatus of claim 1, further comprising an alignment member configured to indicate the second guide port is aligned with the lumen.

10. The apparatus of claim 1, further comprising a fitting connected to the first guide port.

11. The apparatus of claim 1, further comprising a tube connected to the first guide port, the tube configured to receive the cleaning implement and to guide the cleaning implement into the first guide port.

12. The apparatus of claim 1, wherein the guide is a first guide and further comprising at least two guides including the first guide.

13. A system comprising:
the apparatus and the cleaning implement of claim 1; and
an automated cleaning module operatively engaged with the cleaning implement, the brushing module is configured to move the cleaning implement through the conduit.

14. The system of claim 13, further comprising a treatment apparatus comprising the automated cleaning module.

15. A system comprising:
a medical device comprising a valve port in fluid communication with a lumen; and
the apparatus of claim 1, the apparatus is disposed within the valve port of the medical device.

16. The system of claim 15, wherein the lumen comprises a diameter ranging from 0.5 mm to 3 mm.

17. The system of claim 15, wherein the valve port is disposed on a control head of the medical device.

18. The system of claim 15, wherein the valve port is at least one of a gas valve port and a water valve port.

19. A method for cleaning a lumen of a medical device, comprising:
introducing the apparatus of claim 1 into a valve port of the medical device, the valve port is in fluid communication with the lumen of the medical device:
introducing the cleaning implement into the conduit of the apparatus and the lumen of the medical device; and
cleaning the lumen of the medical device with the cleaning implement.

20. The method of claim 19, further comprising contacting the medical device with a detergent.

21. The method of claim 19, further comprising removing a valve from the valve port of the medical device prior to the introducing of the apparatus.

22. The method of claim 19, further comprising:
introducing the apparatus and the medical device in a treatment apparatus; and
operating the treatment apparatus to clean the lumen of the medical device with the cleaning implement.
